(19) United States
(12) Patent Application Publication
(10) Pub. No.: US 2007/0149762 A1
(43) Pub. Date: Jun. 28, 2007

(54) Block Copolypeptide vesicle based materials for drug delivery

(75) Inventor: Daniel Shantz, College Station, TX (US)

Correspondence Address: WINSTEAD SECHREST & MINICK P.C., P.O. BOX 50784, DALLAS, TX, 75201, US

(73) Assignee: Texas A & M University System, College Station, TX (US)

(21) Appl. No.: 11/598,380

(22) Filed: Nov. 13, 2006

Related U.S. Application Data
(60) Provisional application No. 60/736,635, filed on Nov. 15, 2005.
(60) Provisional application No. 60/776,305, filed on Feb. 24, 2006.

(51) Int. Cl. C07K 14/00

(52) U.S. Cl. 530/300

(57) ABSTRACT

In some embodiments, the present invention is directed to block copolypeptides comprising lysine and glycine. In other embodiments, the present invention is directed to a method for reversibly forming supramolecular structures from block copolypeptides. In further embodiments, the present invention is directed to covalently cross-linked supramolecular structures formed from block copolypeptides that comprises lysine and glycine, and a method for producing these covalently cross-linked supramolecular structures.

### TECHNICAL FIELD

The present invention relates generally to block copolypeptides, biomimetic macromolecules based thereon, and a process for preparing these materials. The present invention also relates to structures produced from these biomimetic macromolecules.

### BACKGROUND INFORMATION

Developing new technologies as a consequence of understanding how to control structure, chemistry, and functionality at the nanometer length scale is the ultimate goal of nanotechnology. [Coontz, R., Szuromi, P., Science 2000, 290, 1523-1545]. While chemists, physicists, materials scientists, and engineers have made significant headway in this area, nature is already an expert. In terms of soft matter, one needs to look no further than nature's ability to synthesize macromolecules (proteins) comprised of simple monomer units (amino acids) that have unparalleled structural features on multiple length scales due to primary, secondary, tertiary, and quaternary structure. [Branden, C., Tooze, J., Introduction to Protein Structure; 2nd ed.; Garland: New York, 1999; Creighton, T., Proteins: Structures and Molecular Principles 2nd Edition; W. H. Freeman: New York, 1993]. Perhaps more amazing is the subtlety and elegance of such structures, as replacement of even one amino acid at specific places in the macromolecular sequence can disrupt both local and global structure. This leads to many of the unique properties of proteins/enzymes: exquisite selectivity in binding/catalytically transforming substrates and small (or large) changes in conformation due to very small changes in the local environment (i.e., stimuli such as pH, ions, etc.). The ability to synthesize “biomimetic” soft materials that display these properties would have substantial ramifications for fields as diverse as tissue engineering, molecular electronics, and drug delivery and is an active area of current research. [Allen, T., Cullis, P., Science 2004, 303, 1818-1822; Elbert-Sakiyama, S., Hubbell, J., Annu. Rev. Mater. Res. 2001, 31, 183-201; Hench, L., Polak, J., Science 2002, 295, 1014-1017; Keren, K., Krueger, M., Gilad, R., Ben-Yoseph, G., Sivan, U., Braun, E., Science 2002, 297, 72-75; Langer, R., Tirrell, D., Nature 2004, 428, 487-492; Lutolf, M., Hubbell, J., Nature Biotech. 2005, 23, 47-55].

One possible route to this end is block copolymers. With this motivation, there have been significant advances in the last ten years in the synthesis of “hybrid” block copolymers wherein one block is a poly-α-amino acid and the other a “conventional” polymer (e.g., polystyrene, polybutadiene, PEG) [Babin, J., Rodriguez-Hernandez, J., Lecommandoux, S., Klok, H-A., Achard, M-F., J. Chem. Soc. Faraday Disc. 2005, 128, 179-192; Checot, F., Brulet, A., Oberdisse, J., Gnanou, Y., Mondain-Monval, O., Lecommandoux, S., Langmuir 2005, 21, 4308-4315; Checot, F., Lecommandoux, S., Gnanou, Y., Klok, H-A., Angew. Chem. Int. Ed. 2002, 41, 1339-1343; Checot, F., Lecommandoux, S., Klok, H-A., Gnanou, Y., Eur. Phys. J. E 2003, 10, 25-35; Fukushima, S., Miyata, K., Nishiyama, N., Kanayama, N., Yamasaki, Y., Kataoka, K., J. Am. Chem. Soc. 2005, 127, 2810-2811; Harada, A., Cammas, S., Kataoka, K., Macromolecules 1996, 29, 6183-6188; Harada, A., Kataoka, K., Macromolecules 1995, 28, 5294-5299; Harada, A., Kataoka, K., Macromolecules 1998, 31, 288-294; Harada, A., Kataoka, K., Science 1999, 283, 65-67; Harada, A., Kataoka, K., Macromolecules 2003, 36; Katayose, S., Kataoka, K., Bioconjugate Chem. 1997, 8, 702-707; Kataoka, K., Togawa, H., Harada, A., Yasugi, K., Matsumoto, T., Katayose, S., Macromolecules 1996, 29, 8556-8557; Kataoka, K., Ishihara, A., Harada, A., Miyazaki, H., Macromolecules 1998, 31, 6071-6076; Kukula, H., Schlaad, H., Antonietti, M., Foerster, S., J. Am. Chem. Soc. 2002, 124, 1658-1663; Rodriguez-Hernandez, J., Babin, J., Zappone, B., Lecommandoux, S., Biomacromolecules 2005, 6, 2213-2220; Rodriguez-Hernandez, J., Lecommandoux, S., J. Am. Chem. Soc. 2005, 127, 2026-2027; Klok, H-A., Angew. Chem. Int. Ed. 2002, 41, 1509-1513], as well as block copolypeptides. [Bellomo, E., Davidson, P., Imperor-Clerc, M., Deming, T., J. Am. Chem. Soc. 2004, 126, 9101-9105; Bellomo, E., Wyrsta, M., Pakstis, L., Pochan, D., Deming, T., Nature Materials 2004, 3, 244-248; Breedveld, V., Nowak, A., Sato, J., Deming, T., Pine, D., Macromolecules 2004, 37, 3943-3953; Deming, T., Nature 1997, 390, 386-389; Deming, T., J. Am. Chem. Soc. 1998, 120, 4240-4241; Deming, T., Curtin, S., J. Am. Chem. Soc. 2000, 122, 5710-5717; Pakstis, L., Ozbas, B., Hales, K., Nowak, A., Deming, T., Pochan, D., Biomacromolecules 2004, 5, 312-318; Pochan, D., Pakstis, L., Ozbas, B., Nowak, A., Deming, T., Macromolecules 2002, 35, 5358-5360]. These materials have been shown to self assemble into structures including vesicles, micelles, and hydrogels. [See references previously cited and Nowak, A., Breedveld, V., Pakstis, L., Ozbas, B., Pine, D., Pochan, D., Deming, T., Nature 2002, 417, 424-428; Nowak, A., Breedveld, V., Pine, D., Deming, T., J. Am. Chem. Soc. 2003, 125, 15666-15670]. While these materials are of great interest, the ability to engineer their solution self-assembly behavior is currently lacking. There are many reasons for this, including: 1) most of these materials have a polyelectrolyte block, and 2) many of the structures obtained in solution are kinetic, not thermodynamic, in nature. These characteristics combine such that 1) the materials/structures formed are not amenable to an equilibrium/free energy description, and 2) the structures formed in solution are generally irreversible.

In view of the foregoing, it would be useful to provide block copolypeptides capable of self-assembly into “biomimetic” soft materials, wherein the assembly process is reversible. In addition, it would be desirable to produce structures from these biomimetic materials for applications including drug delivery, controlled released, encapsulation, and biomineralization/biomimetic syntheses of hard matter.

### SUMMARY OF THE INVENTION

In some embodiments, a method of reversibly producing a supramolecular structure, includes introducing free block copolypeptides to an aqueous solution at a first concentration to form the supermolecular structure. The first concentration may be at or above a critical aggregation concentration of the free block copolypeptides in the aqueous solution. Dissociating the supramolecular structure may be accomplished by adjusting the any one or combination of the pH, a salt concentration, and an anion concentration of the aqueous solution.

In some embodiments, a method of producing a covalently cross-linked supramolecular includes introducing free block copolypeptides to an aqueous solution in a concentration above the critical aggregation concentration of the block copolypeptides in the aqueous solution. A second step in the method introduces a cross-linking agent to the aqueous solution.

In some embodiments, a covalently cross-linked supramolecular structure is made from free block copolypeptides and a crosslinking agent. The block copolypeptides includes lysine and glycine.

In yet other embodiments, a reversible supramolecular structure is made from free block copolypeptides. The block copolypeptides includes lysine and glycine.

The foregoing has outlined rather broadly the features of the present invention in order that the detailed description of the invention that follows may be better understood. Additional features and advantages of the invention will be described hereinafter which form the subject of the claims of the invention.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 contains a GPC chromatogram and ¹H NMR spectrum of a Z-Lys₁₂₀-b-Gly₃₀ diblock copolypeptide;

FIG. 2 shows confocal microscopy images of vesicles formed by Lys₁₁₀-b-Gly₅₅ (0.12 mM) and Lys₁₁₀-b-Gly₅₅-b-Lys₁₁₀ (0.06 mM) in solution at pH 7;

FIG. 3 shows a confocal microscopy image of vesicles formed by Lys₂₀₀-b-Gly₅₀ (0.14 mM) conjugated to FITC dye (8% of lysine residue) in solution at pH<7;

FIG. 4 shows a a Zimm plot of Lys₁₁₀-b-Gly₅₅ in water (pH 7; 1M NaBr) obtained at 25° C. over a concentration range of 0.5-3 g/L, where K is an apparatus constant (K=4 πn²(dn/dc)²λ⁴Nₐᵥ) and c is the polymer concentration;

FIG. 5 shows a Zimm plot of Lys₁₁₀-b-Gly₅₅ in water (pH 7; 0.1 M NaBr) obtained at 25° C. over a concentration range of 0.5-3 g/L;

FIG. 6 shows a Zimm plot of Lys₁₁₀-b-Gly₅₅ in water (pH 7; 1 M NaClO₄) obtained at 25° C. over a concentration range of 0.5-3 g/L;

FIG. 7 shows the CD spectra of Lys₁₁₀-b-Gly₅₅ (1.16 μM), Lys₄₀₀-b-Gly₂₀₀ (0.32 μM), Lys₃₄₀-b-Gly-b-₈₅ (0.41 μM), and Lys₁₂₀-b-Gly₃₀ (2.34 μM), below cac, from pH 8.5 to pH 11.2;

FIG. 8 shows the CD spectra of Lys₁₁₀-b-Gly₅₅ (4.05 μM), Lys₄₀₀-b-Gly₂₀₀ (1.12 μM), Lys₃₄₀-b-Gly₈₅ (1.45 μM), and Lys₂₀₀-b-Gly₅₀ (2.46 μM), above cac, from pH 8.5 to pH 11.2;

FIG. 9 shows a confocal microscopy images of vesicles formed by Lys₁₁₀-b-Gly₅₅ (0.12 mM) and Lys₁₁₀-b-Gly₅₅-b-Lys₁₁₀ (0.06 mM) in solution at pH 11;

FIG. 10 shows the CD spectra for the block copolypeptides comprising Lys₁₁₀-b-Gly₅₅ (4.05 μM), Lys₁₁₀-b-Gly₅₅ (1.16 μM), Lys₁₁₀-b-Gly₅₅-b-Lys₁₁₀ (0.59 μM), and Lys₁₁₀-b-Gly₅₅-b-Lys₁₁₀ (2.05 μM), at pH 6, pH 11, and pH 11 after heating both above and below the cac;

FIG. 11 illustrates the circular dichroism (CD) behavior of Lys₁₁₀-b-Gly₅₅ as a function of sodium perchlorate concentration both above and below the cac;

FIG. 12 a schematic illustrating stimuli response behavior of the Lys-b-Gly block copolypeptides; and

FIG. 13 shows the confocal microscopy image of the cross-linked vesicles formed by Lys₁₁₀-b-Gly₅₅-b-Lys₁₁₀ in solution (0.15 mM) at pH 7.

### DETAILED DESCRIPTION OF THE INVENTION

In the following description, specific details are set forth such as specific quantities, sizes, etc. so as to provide a thorough understanding of embodiments of the present invention. However, it will be obvious to those skilled in the art that the present invention may be practiced without such specific details. In many cases, details concerning such considerations and the like have been omitted inasmuch as such details are not necessary to obtain a complete understanding of the present invention and are within the skills of persons of ordinary skill in the relevant art.

In some embodiments, the present invention provides a method of reversibly producing a supramolecular structure. The structure may include, for example, a nominally vesicular structure. Vesicles may result when free block copolypeptides are placed in an aqueous solution at or above a first concentration called the critical aggregation concentration (cac, vide infra). In one embodiment, a vesicular structure may result when using free block copolypeptides that incorporate a hydrophilic portion at one end and a hydrophobic portion at the opposing end. Other supramolecular structures may be formed besides vesicles, for example, micelles and hydrogels.

The formation of the supramolecular structure may be reversible in one embodiment. The dissociation of the structure may be the result of performing at least one of adjusting the pH, adjusting the salt concentration, and adjusting the anion concentration of the aqueous solution. The impact of this adjustment may be raising the cac above the first concentration at which the supramolecular structure was formed. In another embodiment, adjustment of said parameters may reduce the concentration of free block copolypeptides in the aqueous solution to a second concentration which is below the cac. In either case, the result may be the dissociation of the supramolecular structure and release of the free block copolypeptides.

In some embodiments, the present invention provides a method of producing a covalently cross-linked supramolecular structure. The structure may include, for example, a nominally vesicular structure. As described above, vesicular structures may result when placing free block copolypeptides in aqueous solution, wherein one end of the peptide is substantially hydrophilic and the opposing end is substantially hydrophobic. In some embodiments a cross-linking agent is provided which may serve to covalently bond and hold the structure together in its supramolecular form. The nature of the cross-linking agent will depend on the exact amino acids used in the free block copolypeptide sequence. One possible strategy for covalent linkage would be reductive amination using a dialdehyde linker, wherein the aldehyde functional groups may react with side chain amino functional groups of an amino acid such as lysine.

Some embodiments of the present invention relate to the materials made by the methods outlined above and applications of said materials. In biological applications, such as drug delivery, encapsulation, or controlled release, it may be beneficial to incorporate biocompatible blocks into the free block copolypeptide.

Materials

The notation used throughout for the block copolypeptides is Lysₙ-b-Glyₘ, where n and m are the number of amino acids in the respective blocks. Although lysine and glycine were used primarily for production of the copolypeptides described herein, the invention is not so limited and any combination of amino acids may be utilized.

In some embodiments, the free block copolypeptide may include a diblock copolypeptide having the structure Lysₙ-b-Glyₘ, wherein n indicates the number of lysine molecules in the polymer block and may be an integer ranging in value from 30 to 400, and m indicates the number of glycine molecules in the polymer block and comprises an integer ranging in value from 20 to 400. Block copolypeptides incorporating polyglycine may be substantially hydrophobic at this end. One skilled in the art will recognize that other hydrophobic amino acids may be used such as alanine, valine, and leucine. However, peptide blocks of these amino acids may be predisposed to adopting specific conformations, whereas polyglycine may have more available conformations and benefit from greater flexibility. Block copolypeptides incorporating polylysine may be substantially hydrophilic at this end. Because lysine possesses a side chain amino group, the size and structure at this end of the block copolypeptide may be sensitive to a host of conditions including pH, the presence of metal ions, salts, and anion types.

In some embodiments, the free block copolypeptide may include a triblock copolypeptide having the structure Lysₙ-b-Glyₘ-b-Lysₓ, wherein n indicates the number of lysine molecules in the polymer block and may be an integer ranging in value from 20 to 200, m indicates the number of glycine molecules in the polymer block and may be an integer ranging in value from 20 to 100, and x indicates the number of lysine molecules in the polymer block and may be an integer ranging from 20 to 200.

Diblock and triblock copolypeptides may be synthesized by methods known in the art. For example, the synthesis may use N_{ε}-Z-protected L-lysine and Glycine N-carboxyanhydrides (NCAs) using the procedure developed by Deming. [Deming, T. J. Nature 1997, 390, 386-389; Deming, T. J. J. Am. Chem. Soc. 1998, 120, 4240-4241; Deming, T. J.; Curtin, S. A. J. Am. Chem. Soc. 2000, 122, 5710-5717].

Reversible Self Assembly of Supramolecular Structures

In some embodiments, the present invention provides a method of reversibly producing a supramolecular structure based on free block copolypeptides. Dissociation of the structure may be the result of performing at least one of adjusting the pH, adjusting the salt concentration, and adjusting the anion concentration of the aqueous solution. The impact of this adjustment may be raising the cac above the first concentration at which the supramolecular structure was formed. In another embodiment, adjustment of said parameters may reduce the concentration of free block copolypeptides in the aqueous solution to a second concentration which is below the cac. In either case, the result may be the dissociation of the supramolecular structure and release of the free block copolypeptides. In alternate embodiments, the result of adjusting the above parameters may be the change from one supramolecular structure to a different supramolecular structure.

In some embodiments the pH may be adjusted for reversible assembly and disassembly of the supramolecular structure. The side chain amino residues of lysine may be substantially protontated at low pH (below 7) and substantially deprotonated at higher pH (above 7). The degree of protonation may affect whether the supramolecular structure is assembled or disassembled. In another embodiment, the pH may affect the size and/or shape of the supramolecular structure.

In some embodiments, other stimuli may affect the reversible assembly and disassembly of the supramolecular structure, such as the presence of metal ions, anions, and salts. For example, one skilled in the art will recognize that perchlorate anions may induce helix formation in polylysine. Thus, the concentration of perchlorate may affect assembly or disassembly of the supramolecular structure. In alternate embodiments, changing the perchlorate concentration may change the size or shape of the supramolecular structure.

Covalent Cross-Linked Supramolecular Structures

In some embodiments, present invention provides a method of producing a covalently cross-linked supramolecular structure. In this method, one may form a desired supramolecular structure as outline above. By subsequently providing a cross-linking agent, it may be possible to lock the supramolecular structure in place through covalent bond formation. The nature of the cross-linking agent will depend on the exact amino acids used in the free block copolypeptide sequence. One possible strategy for covalent linkage would be reductive amination using a dialdehyde linker, wherein the aldehyde functional groups may react with side chain amino functional groups of an amino acid such as lysine. Alternatively, one may simply use a dialdehyde and covalently link the amino-containing structure by Schiff base formation. In some embodiments, glutaric dialdehdye may be used in cross-linking as well as other commercially available dialdehydes. In alternate embodiments, dialdehydes synthesized by means known in the art, such as the ozonolytic cleavage of cyclic olefins, may be used. One skilled in the art will recognize that a variety of methods exist using difunctional molecules as linkers to generate a stable covalently modified supramolecular structures, and that the use of a dialdehyde is merely exemplary.

Embodiments of the present invention provide reversible and covalently linked supramolecular structures made by the methods described above using free block copolypeptides. In some embodiments, the free block copolypeptide may include a diblock copolypeptide having the structure Lysₙ-b-Glyₘ. In alternate embodiments, the free block copolypeptide may include a triblock copolypeptide having the structure Lysₙ-b-Glyₘ-b-Lysₓ. Supramolecular structure made with these block copolypeptides may be biocompatible and may provide access to multiple structure types including, but not limited to, vesicles, micelles, and hydrogels.

The supramolecular structures generated in accordance with the procedure disclosed herein may be used as agents of drug delivery, controlled release, encapsulation, and biomineralization/biomimetic syntheses of hard matter. For example, one may generate a hollow vesicle for encapsulation by generation of a vesicle with Lysₙ-b-Glyₘ or Lysₙ-b-Glyₘ-b-Lysₓ. Covalent stabilization of the hollow vesicle may be used for controlled release of a drug, for example.

### EXAMPLES

The following examples are included to demonstrate particular embodiments of the present invention. It should be appreciated by those of skill in the art that the methods disclosed in the examples that follow merely represent exemplary embodiments of the present invention. However, those of skill in the art should, in light of the present disclosure, appreciate that many changes can be made in the specific embodiments described and still obtain a like or similar result without departing from the spirit and scope of the present invention.

Diblock and triblock copolypeptides were synthesized using N,Z-protected L-lysine and Glycine N-carboxyanhydrides (NCAs) using the procedure developed by Deming. [Deming, T. J. Nature 1997, 390, 386-389; Deming, T. J. J. Am. Chem. Soc. 1998, 120, 4240-4241; Deming, T. J.; Curtin, S. A. J. Am. Chem. Soc. 2000, 122, 5710-5717]. The polypeptides were then dialyzed and lyophized. ¹H NMR of the protected polymer in d-TFA was used to verify the composition of the polymer, and gel permeation chromatography was used to determine the molecular weight and polydispersity (Styragel HR4 column, effluent: 10 mM LiBr in DMF, Standard: PEO). Below, a detailed description of the synthesis procedures is given for a specific diblock copolypeptide, Lys₁₁₀-b-Gly₅₅.

### Example 1

### Poly(N_{ε}-Z-L-lysine)₁₁₀-b-poly(glycine)₅₅

In a glove box, N_{ε}-Z-L-lysine NCA (1.53 g, 5 mmol) and BpyNiCOD (81 mg, 0.025 mmol) were weighed out, placed in 100 mL flasks and removed from the glove box. Dry THF (˜25 mL for N_{ε}-Z-L-lysine NCA and ˜8 mL for BpyNiCOD) was transferred to the two flasks using a Schlenk line. The BpyNiCOD solution was then transferred to the N_{ε}-Z-L-lysine NCA solution through a cannula under argon atmosphere. Immediately after addition of the BpyNiCOD solution, the flask was stirred for 16 hours. Glycine NCA (0.25 g, 2.5 mmol) was weighed out, dissolved in THF (˜15 mL), and added to the reaction mixture. After stirring for an additional 16 hours, the polypeptide was isolated by adding diethyl ether containing 1 mM HCl to the reaction mixture, causing precipitation of the polypeptide. The polypeptide was dried in vacuo to give a white solid.

### Example 2

### Poly(L-lysine)₁₁₀-b-poly(glycine)₅₅

Poly(N_{ε}-Z-L-lysine)₁₁₀-b-poly(glycine)₅₅ (0.3 g) was dissolved in trifluoroacetic acid (15 mL) in a 100 mL flask. Once all polypeptide was in solution, excess 33 wt % HBr in acetic acid was added via syringe (˜0.9 mL). The solution was left to stir for 20 minutes and then diethyl ether (80 mL) was added to precipitate the polypeptide. The polypeptide was collected via centrifugation, dried in vacuo, and dissolved in deionized water (40 mL). Once the polypeptide was in solution, the solution was transferred to a dialysis tubing cellulose membrane (Sigma, MWCO 12,400) and the membrane was placed in a 10 L container of deionized water. The water was exchanged 2-3 times per day over the next three days. The solution was then placed on a freeze dryer to yield the product as a white spongy material. Typical polypeptide yields were between 75% and 85% based on the NCA.

### Example 3

### FITC Labeling Procedure

Block copolypeptides conjugated with fluorescein (FITC) through the N_{ε}-amino group were prepared as follows. A 1 mL solution of Lys₂₀₀-b-Gly₅₀ (12 mg/mL) in sodium bicarbonate buffer (0.1 M, pH 8.3) was prepared and to it 0.4 mL of 10 mg/mL fluorescein-5-EX, succinimidyl ester (Molecular Probes) in dry DMF was added. After 60 minutes of vigorous stirring, the solution was transferred to a dialysis tubing cellulose membrane (Sigma, MWCO 12,400) and the membrane was placed in a 1 L container of deionized water. The solution was dialyzed for 24 hours and then used for characterization.

### Example 4

### Cross-Linked Vesicles

Covalently cross-linked vesicles were prepared as follows: a 1 wt % solution of glutaric dialdehyde was prepared from 50 wt % glutaric dialdehyde in water. To a stirred solution of vesicles (20 mL, 66 mg of Lys₁₁₀-b-Gly₅₅-b-Lys₁₁₀) was added a freshly prepared solution of glutaric dialdehyde (0.3 ml, 1 wt %). After 15 minutes of vigorous stirring, the solution was transferred to a dialysis tubing cellulose membrane (Sigma, MWCO 12,400) and the membrane was placed in a 1 L container of deionized water. The solution was dialyzed for 24 hours and then used for characterization.

Characterization

Gel permeation chromatography measurements were performed before deprotection of the polypeptides using a Shimadzu system consisting of one Styragel HR4 (Waters) column, eluted with 10 mM LiBr in DMF at 25° C. The eluent flow rate was 1 mL/min. Calibration was performed using poly(ethylene oxide) standards. ¹H NMR spectra were recorded at 300 MHz on a Mercury 300 Varian spectrometer using d-TFA as solvent. Critical aggregation concentrations (cac) were determined by measuring the conductivity of block copolypeptide solutions as a function of polypeptide concentration (0.001 to 1 mg/mL). The conductivity measurements were performed with an Accumet AB30 conductivity meter with a conductivity cell (0.1 cm⁻¹ cell constant). The calculated molecular weight (Table 1, below) was used to convert to molar concentrations.

TABLE 1

| Copolypeptide | Calculated Molecular Weight |
|---|---|
| Lys₁₂₀Gly₃₀ | 17.1K |
| Lys₂₀₀Gly₅₀ | 28.5K |
| Lys₃₄₀Gly₈₅ | 48.4K |
| Lys₁₁₀Gly₅₅ | 17.3K |
| Lys₃₂₀Gly₁₆₀ | 50.1K |
| Lys₄₀₀Gly₂₀₀ | 62.6K |
| Lys₂₈Gly₂₈Lys₂₈ | 8.76K |
| Lys₄₈Gly₁₂Lys₄₈ | 13.0K |
| Lys₁₂₀Gly₃₀Lys₁₂₀ | 32.4K |
| Lys₁₆₀Gly₄₀Lys₁₆₀ | 43.2K |
| Lys₁₁₀Gly₅₅Lys₁₁₀ | 34.1K |

Dynamic light scattering measurements were carried out using a Brookhaven ZetaPALS instrument. Vertically polarized light with a wavelength of 658 nm was used as the incident beam. The intensity of the scattered light was measured at a 90° scattering angle and temperature controlled at 25° C. Time-averaged particle size distributions were collected over an analysis time of at least 10 min. The hydrodynamic diameter (Rₕ) of the polypeptide supramolecular structures was determined from light scattering experiments using Brookhaven Instruments Dynamic light scattering software. Static light scattering (SLS) measurements and dynamic light scattering (DLS) measurements at different angles were performed with a Brookhaven Instruments BI-200SM goniometer using a Melles Griot HeNe laser with a wavelength of 632.8 nm. Measurements were performed on block copolypeptide solutions (2 mg/mL) that were sonicated for 30 minutes. The pH was adjusted using 0.01 to 0.5 N NaOH and/or HCl solutions. Circular dichroism (CD) measurements were performed with a AVIV Stopped Flow Circular Dichroism Spectrometer Model 202SF. CD measurements were performed on block copolypeptide solutions above and below the cac, using 0.01 to 0.5 N NaOH solutions to adjust the pH to keep the block copolypeptide concentrations within the desired range. Confocal images were obtained with a Carl Zeiss LSM 5 PASCAL inverted confocal microscope equipped with a 63× oil immersion objective (NA=1.4). The laser excitation wavelength was 543 nm. Samples were mounted on glass slides and a dilute rhodamine 6G solution (40 μM) was used as the fluorescent agent. For block copolypeptides covalently conjugated to fluorescence, the laser excitation wavelength of 488 nm was chosen for FITC (λ_{Ex}=494 nm, λ_{Em}=518 nm).

Table 2 summarizes the block copolypeptide molecular weights and polydispersity as determined by GPC and ¹H NMR.

TABLE 2

| Copolypeptide | Mₙ | M_{w}/Mₙ |
|---|---|---|
| Z-Lys₁₂₀Gly₃₀ | 35,600 | 1.21 |
| Z-Lys₂₀₀Gly₅₀ | 53,500 | 1.17 |
| Z-Lys₃₄₀Gly₈₅ | 95,300 | 1.24 |
| Z-Lys₁₁₀Gly₅₅ | 31,800 | 1.20 |
| Z-Lys₃₂₀Gly₁₆₀ | 93,800 | 1.30 |
| Z-Lys₄₀₀Gly₂₀₀ | 118,200 | 1.09 |
| Z-Lys₄₈Gly₁₂Z-Lys₄₈ | 26,100 | 1.28 |
| Z-Lys₁₂₀Gly₃₀Z-Lys₁₂₀ | 64,500 | 1.17 |
| Z-Lys₁₆₀Gly₄₀Z-Lys₁₆₀ | 79,300 | 1.30 |
| Z-Lys₁₁₀Gly₅₅Z-Lys₁₁₀ | 59,000 | 1.06 |

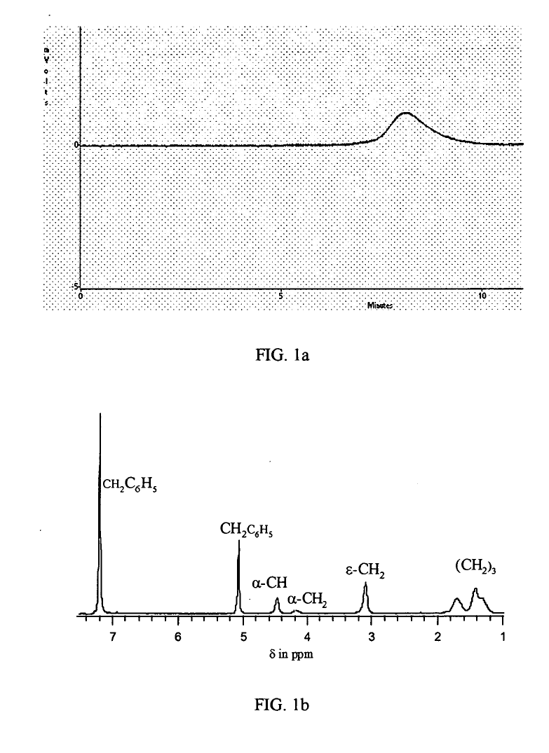

As shown in previous work, [Deming, T., Nature 1997, 390, 386-389], the current synthesis methodology leads to polypeptides possessing a reasonably narrow molecular weight distribution. A representative GPC chromatogram and ¹H NMR spectrum of Z-Lys₁₂₀-b-Gly₃₀ diblock copolypeptide is shown in FIG. 1. ¹H NMR of the polypeptide in d-TFA was performed to determine the compositions of the block copolypeptides, which were found to be within 5% of predicted values based on the ratio of NCAs. The block ratio of Z-L-lysine and glycine block was determined from the ratio of the peak intensity of α-hydrogen protons of glycine (CH₂: δ=4.2 ppm) and benzyl protons of Z-L-lysine (COOCH₂C₆H₅: δ=7.2 ppm) After deprotection the remaining Z groups in the block copolypeptides were below 3% as determined by ¹H NMR.

### Example 5

### Block Copolypeptide Solution Behavior at pH 7

Table 3 lists block copolypeptides synthesized, their critical aggregation concentration (cac) at pH 7 and the hydrodynamic radii (Rₕ)of the supramolecular objects (vesicles) formed determined using dynamic light scattering (measured in buffer).

TABLE 3

| Copolypeptide | cac (μM) | Rₕ (nm) |
|---|---|---|
| Lys₁₂₀-b-Gly₃₀ | 5.84 | 316 |
| Lys₂₀₀-b-Gly₅₀ | 1.4 | 383 |
| Lys₃₄₀-b-Gly₈₅ | 0.83 | 950 |
| Lys₁₁₀-b-Gly₅₅ | 1.73 | 302 |
| Lys₃₂₀-b-Gly₁₆₀ | 0.8 | 580 |
| Lys₄₀₀-b-Gly₂₀₀ | 0.48 | 635 |
| Lys₄₈-b-Gly₁₂-b-Lys₄₈ | 3.08 | 176 |
| Lys₁₂₀-b-Gly₃₀-b-Lys₁₂₀ | 1.23 | 572 |
| Lys₁₆₀-b-Gly₄₀-b-Lys₁₆₀ | 0.58 | 745 |
| Lys₁₁₀-b-Gly₅₅-b-Lys₁₁₀ | 0.88 | 534 |

The table shows that the polypeptides form supramolecular structures at low concentrations in solution, and the cac values are well defined based on conductivity measurements. For a fixed block ratio of approximately 4 Lys:1 Gly (e.g. Lys₁₂₀-b-Gly₃₀, Lys₂₀₀-b-Gly₅₀, and Lys₃₄₀-b-Gly₈₅) increasing the molecular weight leads to larger vesicles. For an approximately fixed molecular weight increasing the Lys:Gly ratio also leads to larger vesicles.

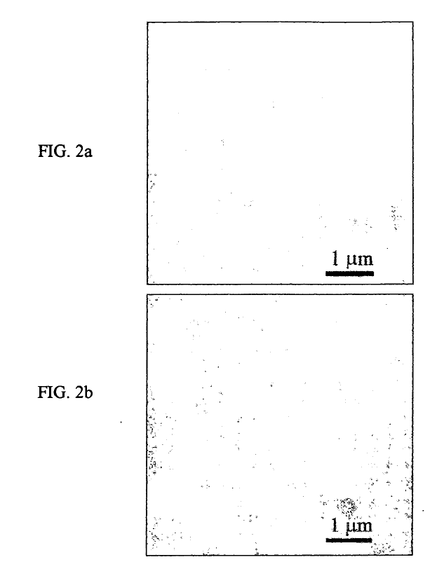

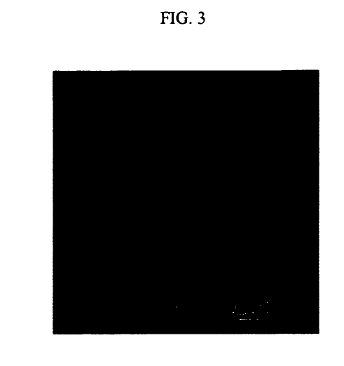

The nature of the microstructures formed in solutions by the block copolypeptides was probed using confocal microscopy, conductivity, and dynamic light scattering (DLS) measurements. FIG. 2 shows confocal microscopy images of vesicles formed by Lys₁₁₀-b-Gly₅₅ (0.12 mM) (top), and Lys₁₁₀-b-Gly₅₅-b-Lys₁₁₀ (0.06 mM) (bottom), in solution at pH 7. The ring structures without the observed fluorescence (dark region) indicates the microstructures are vesicles. Also, the core region of the vesicles exhibits fluorescence, which is consistent with the presence of vesicles. Further evidence for the presence of vesicles as compared to micelles is based on confocal images of Lys-b-Gly block copolypeptides where a small fraction of the lysine side chains (8%) have been labeled with FITC, a fluorophore. FIG. 3 shows a confocal microscopy image of vesicles formed by Lys₂₀₀-b-Gly₅₀ (0.14 mM) conjugated to FITC dye (8% of lysine residue) in solution at pH<7. This is additional proof that the Lys-b-Gly block copolypeptides self-assemble into vesicles based on the observed fluorescence from the shell. Finally, based on the contour lengths calculated for the polypeptide chains and the size of the microstructures in solution it is concluded that all block copolypeptides form vesicles. Confocal microscopy, dynamic light scattering, and contour length calculations are all consistent with the presence of polypeptide vesicles. Further, it should be noted that vesicle formation is reversible, i.e., dilution below the cac leads to free polypeptide chains in solution, and addition of polypeptide to above the cac leads to vesicles of the same size. Block copolypeptide solutions stored over three weeks were measured using DLS and the results are identical to that of freshly prepared solutions, indicating the vesicles are stable at pH 7. DLS measurements performed at several scattering angles (30°, 45°, 60°, 90°, 120°, 135°, and 150°) are consistent with spherical objects, and the hydrodynamic diameters determined at the different angles are all within 3% of one another. The identical DLS results were obtained whether or not the solutions had been sonicated.

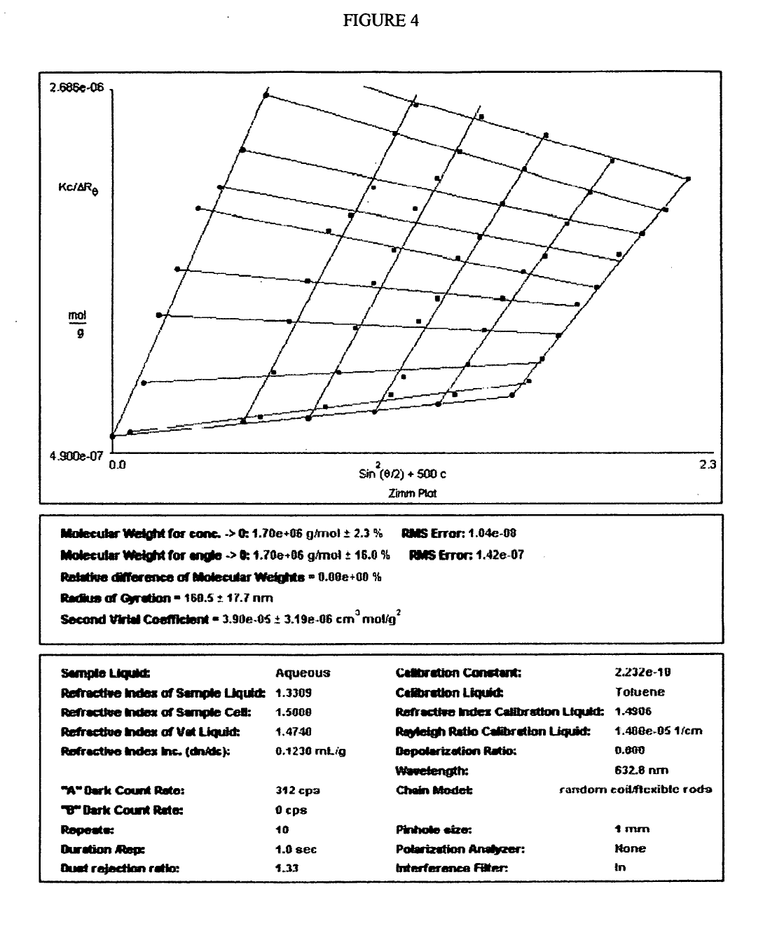

The large vesicle sizes are observed since the lysine block is highly extended at pH 7, as the side-chain N_{ε}-amine groups are protonated. To probe this, DLS measurements were performed on mixtures with varying electrolyte content. The vesicle size is strongly sensitive to ionic strength, consistent with this picture. As an example, for Lys₁₁₀-b-Gly₅₅ (0.12 mM) in only buffer solution, the vesicle hydrodynamic radius is 302 nm, whereas it decreased to 239 nm, 179 nm, and 165 nm in 0.005 M, 0.1 M, and 1 M NaBr, respectively. Confocal microscopy on a solution of vesicles containing 0.1 M NaBr indicates the objects in solution are still vesicles. Further evidence for the formation of vesicles by Lys₁₁₀-b-Gly₅₅ in the presence of NaBr is obtained from static light scattering (SLS). For the Lys₁₁₀-b-Gly₅₅ system the radius of gyration (R_{g}) is 161 nm at pH 7 in 1 M NaBr. This is shown in FIG. 4, which is a Zimm plot of Lys₁₁₀-b-Gly₅₅ in water (pH 7; 1M NaBr) obtained at 25° C. over a concentration range of 0.5-3 g/L, where K is an apparatus constant (K=4; πn² (dn/dc)²λ⁴Nₐᵥ) and c is the polymer concentration.

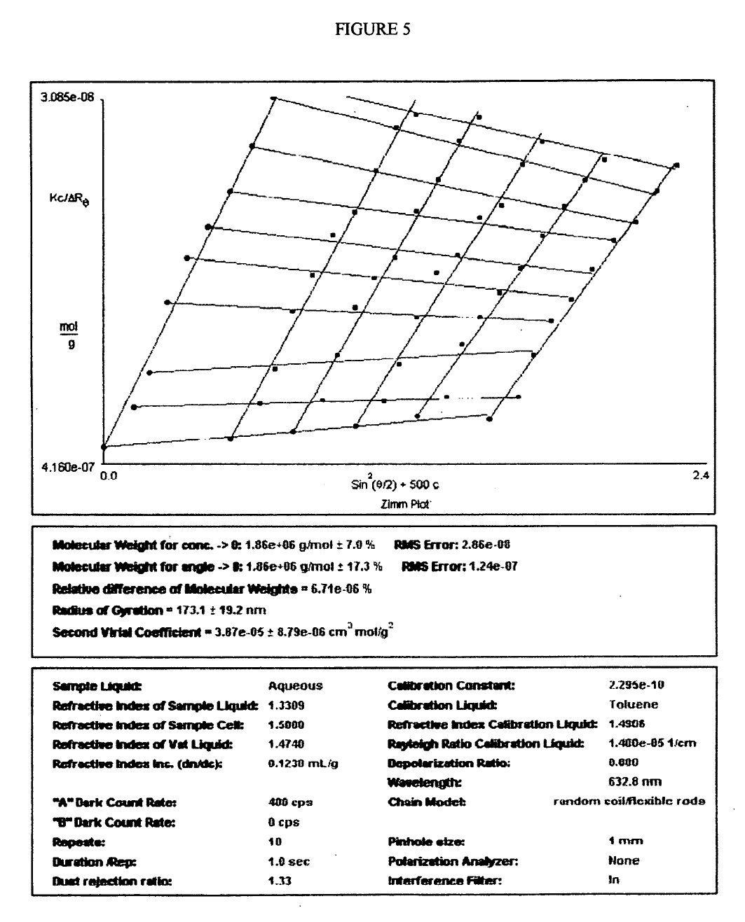

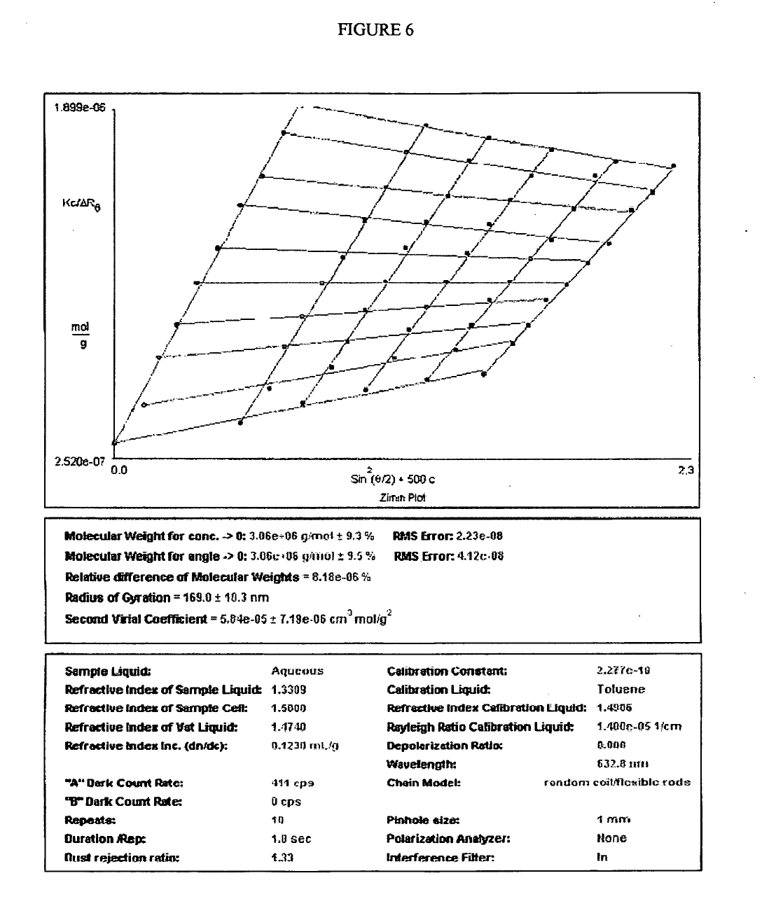

The ratio of R_{g} to R_{H} (ρ) can be used to ascertain the microstructure formed (i.e., vesicles, ρ=1 versus micelles, ρ=0.78). [Checot, F., Lecommandoux, S., Klok, H-A., Gnanou, Y., Eur. Phys. J. E 2003, 10, 25-35]. The value of ρ is 0.98 for the mixtures at pH 7 in 1 M NaBr. This indicates the objects formed at pH 7 in 1 M NaBr are vesicles. A series of measurements were also performed at different ionic strengths. In all cases the ρ value is between 0.95-0.98, consistent with the presence of vesicles as shown in FIG. 4, as well as FIG. 5 (Zimm plot of Lys₁₁₀-b-Gly₅₅ in water (pH 7; 0.1 M NaBr) obtained at 25° C. over a concentration range of 0.5-3 g/L) and FIG. 6 (Zimm plot of Lys₁₁₀-b-Gly₅₅ in water (pH 7; 1 M NaClO₄) obtained at 25° C. over a concentration range of 0.5-3 g/L). These results show that the size of these objects is sensitive to ionic strength, allowing one to adjust the vesicle size (or at least the corona) in a deliberate manner.

### Example 6

### Block Copolypeptide Solution Behavior at High pH

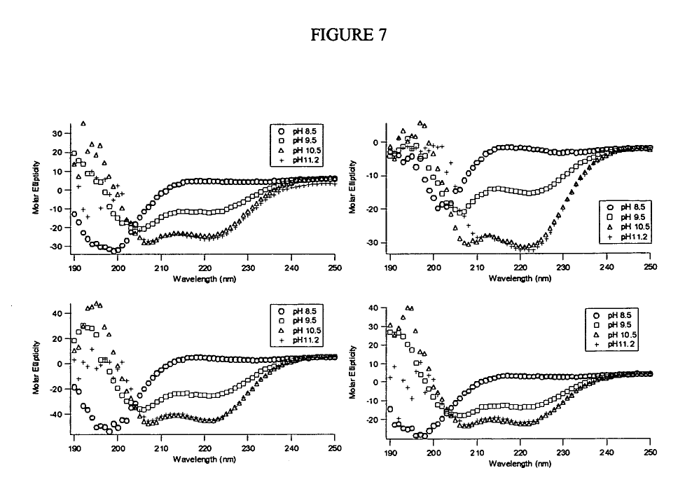

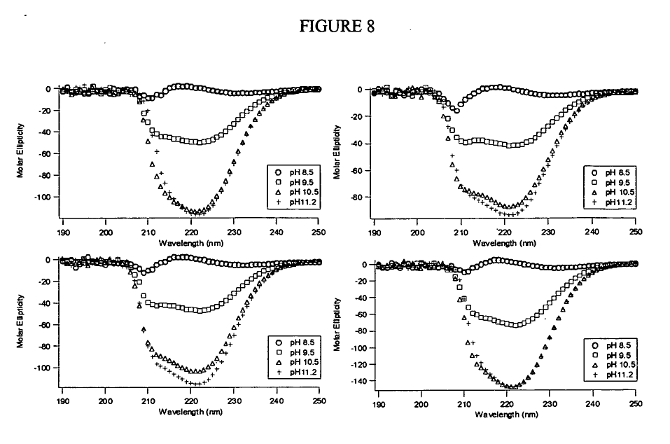

Another feature of these macromolecules that could be used to modulate the supramolecular architectures formed in solution is the folded state of the poly-L-lysine block. With that in mind, FIGS. 7 and 8 show the CD behavior of several block copolypeptides as a function of pH both above and below the cac. FIG. 7 shows the CD spectra of (clockwise from top left) Lys₁₁₀-b-Gly₅₅ (1.16 μM), Lys₄₀₀-b-Gly₂₀₀ (0.32 μM), Lys₃₄₀-b-Gly-b-₈₅ (0.41 μM), and Lys₁₂₀-b-Gly₃₀ (2.34 μM) from pH 8.5 to pH 11.2 (below cac.). FIG. 8 shows the CD spectra of (clockwise from top left) Lys₁₁₀-b-Gly₅₅ (4.05 μM), Lys₄₀₀-b-Gly₂₀₀ (1.12 μM), Lys₃₄₀-b-Gly₈₅ (1.45 μM), and Lys₂₀₀-b-Gly₅₀ (2.46 μM) from pH 8.5 to pH 11.2 (above cac). Several points are of note. These polypeptides are model systems for circular dichroism as the glycine blocks are “invisible” by CD because they lack a chiral center. Below the cac a clear random coil→α-helix transition is observed at pH 9.5. By contrast, CD measurements above the cac show that at higher pH the chains adopt a mixture of α-helix and β-sheet conformations. Consistent with this result the size of the supramolecular object at pH 11 is much smaller as determined by DLS, as shown in Table 4 below.

TABLE 4

| Copolypeptide | Rₕ (nm) |
|---|---|
| Lys₁₂₀-b-Gly₃₀ | 127 |
| Lys₂₀₀-b-Gly₅₀ | 165 |
| Lys₃₄₀-b-Gly₈₅ | 320 |
| Lys₁₁₀-b-Gly₅₅ | 175 |
| Lys₄₈-b-Gly₁₂-b-Lys₄₈ | 76 |
| Lys₁₂₀-b-Gly₃₀-b-Lys₁₂₀ | 162 |
| Lys₁₆₀-b-Gly₄₀-b-Lys₁₆₀ | 237 |
| Lys₁₁₀-b-Gly₅₅-b-Lys₁₁₀ | 216 |

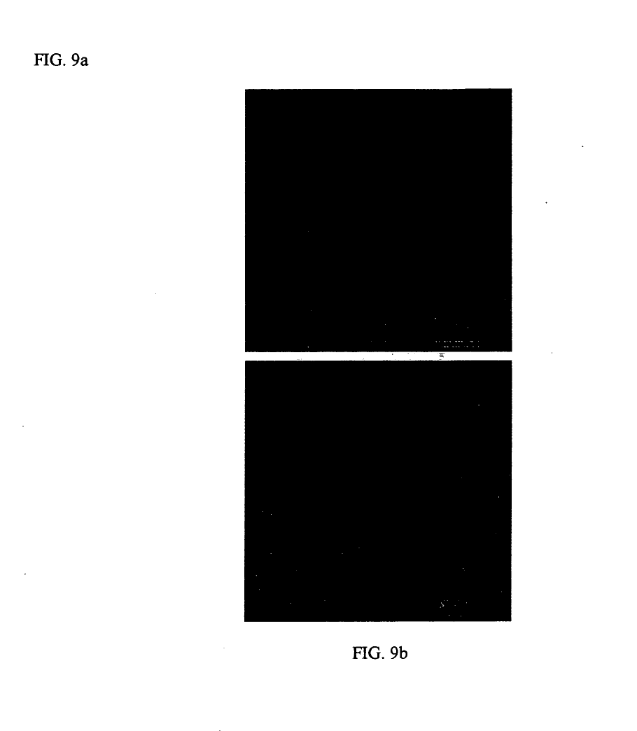

As an example, for the Lys₁₁₀-b-Gly₅₅ diblock copolypeptides the hydrodynamic radius of the supramolecular objects decreased from 302 nm at pH 7 to 175 nm at pH 11, an approximately 40% decrease. FIG. 9 shows confocal microscopy images of vesicles formed by Lys₁₁₀-b-Gly₅₅ (0.12 mM) (top), and Lys₁₁₀-b-Gly₅₅-b-Lys₁₁₀ (0.06 mM) (bottom), in solution at pH 11. The size determined from these confocal images is in good agreement with the DLS results. Given that the resolution of the scanning confocal microscope used is approximately λ/4 (˜120 nm), it is not possible to ascertain whether the microstructures formed at pH 11 are micelles or vesicles. Given that the radius of gyration values (R_{g}) are larger than the values of the contour lengths of the chains (contour length ˜50-60 nm), it seems likely the objects are not micelles. Preliminary TEM results are consistent with the presence of vesicles. Most interesting is that this response to pH is completely reversible as indicated by DLS and confocal images. Numerous cycles of varying the pH between 7 and 11 indicate that these transitions are completely reversible (i.e., DLS results from cycle 1 is the same as the results after several pH cycles). Further the occurrence of these size changes clearly correlate to the conformational changes of the poly-L-lysine block. As such it seems likely the changes observed are due to conformational changes of the lysine block. Given that polyglycine is known to be highly insoluble in water [Qu, Y., Bolen, C., Bolen, D., Proc. Natl. Acad. Sci. USA 1998, 95, 9268-9273; Robinson, D., Jencks, W., J. Am. Chem. Soc. 1965, 87, 2462-2470], it seems likely that the conformational changes of the poly-L-lysine block are primarily responsible for the change in vesicle size. Experiments performed in our lab studying polyglcyine 20-50 residues in length are consistent with previous work and show their poor solubility in water. Also noteworthy is that for the highest molecular weight diblocks (Lys₃₂₀-b-Gly₁₆₀ and Lys₄₀₀-b-Gly₂₀₀), the supramolecular objects at pH 11 precipitate out of the solution immediately, however the objects still retain their spherical shape (not shown). For other block polypeptides, the supramolecular objects formed by block copolypeptides with higher molecular weight tend to precipitate out of the solution sooner than those with lower molecular weight. Other disturbances, such as heating or sonication, can also cause precipitation. However, it is important to note that changing the pH back to 6 results in dissolution of the objects and the formation of vesicles.

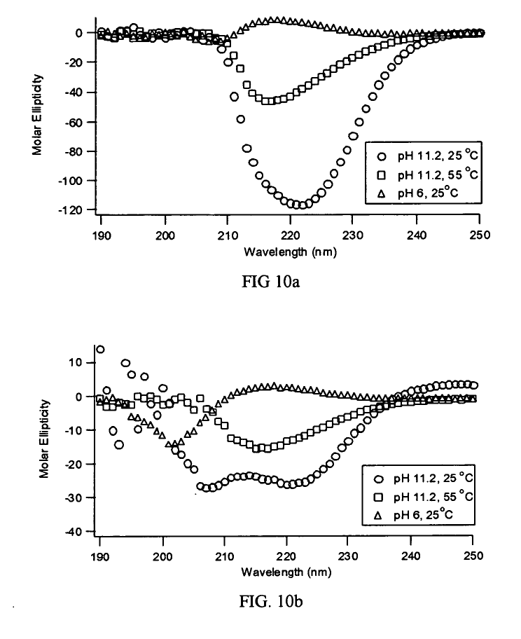

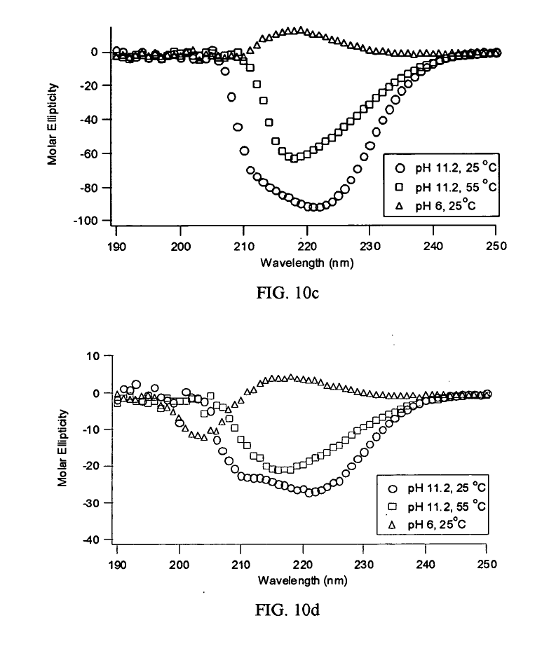

The CD results at pH 11 are somewhat ambiguous; to probe these further a solution at pH 11 was heated up to 328° K, where the helix→sheet transition will rapidly occur. FIG. 10 shows the CD spectra for the block copolypeptides at pH 6, pH 11, and pH 11 after heating both above and below the cac. (clockwise from top left, Lys₁₁₀-b-Gly₅₅ (4.05 μM), Lys₁₁₀-b-Gly₅₅ (1.16 μM), Lys₁₁₀-b-Gly₅₅-b-Lys₁₁₀ (0.59 μM), and Lys₁₁₀-b-Gly₅₅-b-Lys₁₁₀ (2.05 μM)). The heating does in fact lead to the clear formation of the β-sheet structure based on CD as a single minimum is observed at 216 nm. Again, this transition is completely reversible, as cooling the mixture to room temperature and changing the pH back to 6 results in the conversion of the β-sheet structure to a random coil. Also noteworthy is that heating the supramolecular objects formed by Lys₁₁₀-b-Gly₅₅ (0.12 mM) at pH 11 caused them to aggregate and ultimately precipitate out of the solution, however the objects still retain their spherical shape (not shown). Again, cooling the mixture to room temperature and changing the pH back to 6 results in the formation of vesicles.

### Example 7

### Stimuli other than pH

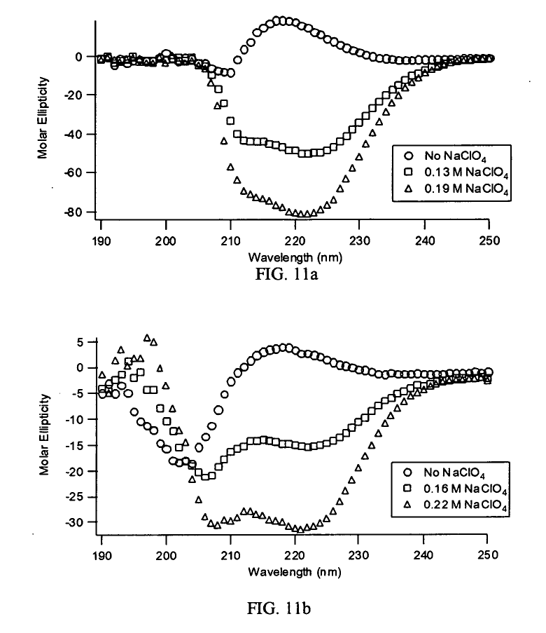

The work above indicates that both ionic strength and pH can be used to manipulate the size and structures of the supramolecular objects formed by Lys-b-Gly polypeptides and that these changes are reversible. Additionally, other stimuli were investigated. Experiments were performed in the presence of perchlorate anions that are known to induce helix-formation in poly-L-lysine at pH 7. [Daly, W., Poché, D., Tetrahedron Lett. 1988, 29, 5859-5862]. FIG. 11 shows the circular dichroism (CD) behavior of Lys₁₁₀-b-Gly₅₅ as a function of sodium perchlorate concentration both above and below the cac (Lys₁₁₀-b-Gly₅₅ (4.05 μM) (top), and Lys₁₁₀-b-Gly₅₅ (1.16 μM) (bottom), at different sodium perchlorate concentrations). The presence of perchlorate anions does in fact lead to the clear formation of the α-helix structure below the cac based on CD as the double minima are observed at 208 and 222 nm. By contrast, CD measurements above the cac show that in the presence of perchlorate anions the chains adopt a mixture of α-helix and β-sheet conformations. The CD results clearly show that the lysine block adopts different folded states above and below the cac. Static light scattering (SLS) measurements were also performed on solutions above the cac at pH 7 in 1 M NaClO₄. As an example, for the Lys₁₁₀-b-Gly₅₅ system the radius of gyration (R_{g}) is 169 nm at pH 7 in 1 M NaClO₄ (FIG. 6). The hydrodynamic radius (Rh) for the same polymer at pH 7 in 1 M NaClO₄ is 155 nm. The value of ρ is 1.09 for the mixtures at pH 7 in 1 M NaClO₄. It is not clear how the microstructures with the secondary structure adopted by the poly-L-lysine block in the corona can impact the ρ value. However, the value indicates that the objects formed at pH 7 in 1 M NaClO₄ are vesicles and it is consistent with TEM analysis (not shown). Ions other than perchlorate may be employed to manipulate the size and structures of the supramolecular objects formed by polypeptides.

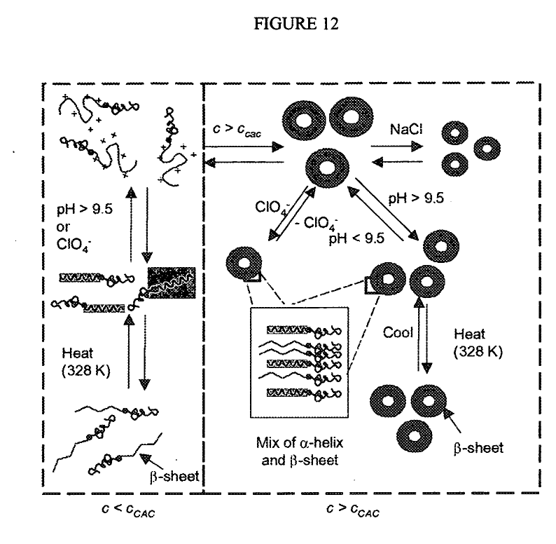

In summary, lysine-glycine block copolypeptides form soft materials that are responsive to a variety of stimuli including pH, electrolyte, and temperature, and this correlates with conformational changes of the lysine block. Most significant is that every transition shown is completely reversible. FIG. 12 is a schematic illustrating stimuli response behavior of the Lys-b-Gly block copolypeptides and summarizes the solution behavior of these macromolecules.

### Example 8

### Translating Non-Covalent Structures into Covalent Soft Matter

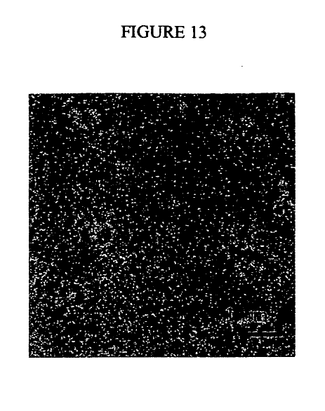

Given the interest in using biological macromolecules or mimics thereof either as components of nanostructured materials or as templates to assemble such materials we report here preliminary studies in this vein. One point of interest is converting the non-covalent vesicles formed at pH 7 into covalent, hollow, spheres. This was achieved by simply using glutaric dialdehyde to cross-link the lysine blocks. [Rodriguez-Hernandez, J., Babin, J., Zappone, B., Lecommandoux, S., Biomacromolecules 2005, 6, 2213-2220]. The molar ratio between glutaric dialdehyde and Lys₁₁₀-b-Gly₅₅-b-Lys₁₁₀ block copolypeptide is approximately 15:1 (n_{Aldehyde}:n_{side chain amines}=3:22). FIG. 13 shows the confocal microscopy image of the cross-linked vesicles formed by Lys₁₁₀-b-Gly₅₅-b-Lys₁₁₀ in solution (0.15 mM) at pH 7. According to DLS and confocal microscopy measurements, the size and shape of the cross-linked vesicles (Rₕ=412 nm) are comparable to the parent vesicles, indicating that the information content of the supramolecular objects in solution can be translated into nanostructured covalent soft matter. DLS measurements were performed on mixtures with varying pH of the solution and the hydrodynamic radii of the supramolecular objects decrease in size to 215 nm at pH 11, an approximately 50% decrease. DLS measurements were also performed on mixtures with varying electrolyte content. The vesicle size is strongly sensitive to ionic strength. For Lys₁₁₀-b-Gly₅₅-b-Lys₁₁₀ (0.06 mM), the hydrodynamic radius decreased to 235 nm and 151 nm in 0.01 M and 0.1 M NaBr, respectively. This last result demonstrates that these materials can be converted from non-covalent to covalent supramolecular structures yet are still sensitive to stimuli. In addition to glutaric dialdehyde, suitable cross-linking agents include, but are not limited to, any dialdehyde or other compound that would readily react with a primary amine, such as a diacid chloride.

Here, the self-organization of poly(L-lysine)-b-polyglycine diblock and triblock copolypeptides in aqueous solution is reported. These block copolypeptides can self-assemble into vesicles at neutral, acidic, and basic condition. The structures formed in solution by these copolypeptides are investigated using, inter alia, confocal microscopy and dynamic light scattering. The secondary structure adopted by the lysine block at different solution conditions is characterized by circular dichroism (CD). Three major conclusions result from the current work: (1) lysine-b-glycine block copolypeptides form vesicles, and their size can be manipulated based on solution conditions, (2) the stimuli to induce these changes can be pH, salt, or an anion (including, but not limited to, perchlorate anion), and (3) these structural transitions are shown to be primarily due to the change in conformation of the lysine block, and are completely reversible. It is also demonstrated that non-covalent supramolecular structures formed can be converted to covalent supramolecular structures and their swelling behavior can be regulated by changes in the pH or ionic strength. The results of this work show that these previously unreported materials have unique properties of relevance to numerous applications including drug delivery, controlled released, encapsulation, and biomineralization/biomimetic syntheses of hard matter.

All publications referenced herein are hereby incorporated by reference. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed is:

1. A method of reversibly producing a supramolecular structure comprising free block copolypeptides, the method comprising:
introducing free block copolypeptides into an aqueous solution at a first concentration to form the supermolecular structure;
wherein the first concentration is above a critical aggregation concentration of the free block copolypeptides in the aqueous solution; and
dissociating the supramolecular structure by adjusting at least one parameter selected from pH, a salt concentration and an anion concentration of the aqueous solution.

2. The method of claim 1, wherein the adjusting of at least one parameter raises the critical aggregation concentration above the first concentration of the free block copolypeptides.

3. The method of claim 1, wherein the adjusting of at least one parameters reduces the concentration of free block copolypeptides in the aqueous solution to a second concentration;
wherein the second concentration is below the critical aggregation concentration of the free block copolypeptides.

4. The method of claim 1, wherein the free block copolypeptide comprises a diblock copolypeptide having the structure Lysₙ-b-Glyₘ, wherein n indicates the number of lysine molecules in the polymer block and comprises an integer ranging in value from 30 to 400, and m indicates the number of glycine molecules in the polymer block and comprises an integer ranging in value from 20 to 400.

5. The method of claim 1, wherein the free block copolypeptide comprises a triblock copolypeptide having the structure Lysₙ-b-Glyₘ-b-Lysₓ, wherein n indicates the number of lysine molecules in the polymer block and comprises an integer ranging in value from 20 to 200, m indicates the number of glycine molecules in the polymer block and comprises an integer ranging in value from 20 to 100, and x indicates the number of lysine molecules in the polymer block and is an integer having a value of from 20 to 200.

6. A method of producing a covalently cross-linked supramolecular structure comprising free block copolypeptides, the method comprising:
introducing free block copolypeptides to an aqueous solution in a concentration above the critical aggregation concentration of the block copolypeptides in the aqueous solution; and introducing a cross-linking agent to the aqueous solution.

7. The method of claim 6, wherein the free block copolypeptide comprises a diblock copolypeptide having the structure Lysₙ-b-Glyₘ, wherein n indicates the number of lysine molecules in the polymer block and comprises an integer ranging in value from 30 to 400, and m indicates the number of glycine molecules in the polymer block and comprises an integer ranging in value from 20 to 400.

8. The method of claim 6, wherein the free block copolypeptide comprises a triblock copolypeptide having the structure Lysₙ-b-Glyₘ-b-Lysₓ, wherein n indicates the number of lysine molecules in the polymer block and comprises an integer ranging in value from 20 to 200, m indicates the number of glycine molecules in the polymer block and comprises an integer ranging in value from 20 to 100, and x indicates the number of lysine molecules in the polymer block and is an integer having a value of from 20 to 200.

9. A covalently cross-linked supramolecular structure, the structure comprising:
free block copolypeptides: and
a crosslinking agent;
wherein the block copolypeptides comprise lysine and glycine.

10. The structure of claim 9, wherein the free block copolypeptide comprises a diblock copolypeptide having the structure Lysₙ-b-Glyₘ, wherein n indicates the number of lysine molecules in the polymer block and comprises an integer ranging in value from 30 to 400, and m indicates the number of glycine molecules in the polymer block and comprises an integer ranging in value from 20 to 400.

11. The structure of claim 9, wherein the free block copolypeptide comprises a triblock copolypeptide having the structure Lysₙ-b-Glyₘ-b-Lysₓ, wherein n indicates the number of lysine molecules in the polymer block and comprises an integer ranging in value from 20 to 200, m indicates the number of glycine molecules in the polymer block and comprises an integer ranging in value from 20 to 100, and x indicates the number of lysine molecules in the polymer block and is an integer having a value of from 20 to 200.

12. The structure of claim 9, wherein the supramolecular structure is used for at least one selected from drug delivery, controlled release, encapsulation, and biomineralization/biomimetic syntheses of hard matter.

13. A reversible supramolecular structure, the structure comprising:
free block copolypeptides;
wherein the block copolypeptides comprise lysine and glycine.

14. The structure of claim 13, wherein the free block copolypeptide comprises a diblock copolypeptide having the structure Lysₙ-b-Glyₘ, wherein n indicates the number of lysine molecules in the polymer block and comprises an integer ranging in value from 30 to 400, and m indicates the number of glycine molecules in the polymer block and comprises an integer ranging in value from 20 to 400.

15. The structure of claim 13, wherein the free block copolypeptide comprises a triblock copolypeptide having the structure Lysₙ-b-Glyₘ-b-Lysₓ, wherein n indicates the number of lysine molecules in the polymer block and comprises an integer ranging in value from 20 to 200, m indicates the number of glycine molecules in the polymer block and comprises an integer ranging in value from 20 to 100, and x indicates the number of lysine molecules in the polymer block and is an integer having a value of from 20 to 200.

16. The structure of claim 13, wherein the supramolecular structure is used for at least one selected from drug delivery, controlled release, encapsulation, and biomineralization/biomimetic syntheses of hard matter.
